(19) United States
(12) Patent Application Publication
(10) Pub. No.: US 2021/0369235 A1
(43) Pub. Date: Dec. 2, 2021

(54) METHOD FOR DETERMINING PULSE TRANSMISSION TIME, ARTERIOSCLEROSIS DETECTION APPARATUS AND SYSTEM

(71) Applicant: BOE TECHNOLOGY GROUP CO., LTD., BEIJING (CN)

(72) Inventors: Hui DU, BEIJING (CN); Jinye LIU, BEIJING (CN); Shuai CAO, BEIJING (CN)

(73) Assignee: BOE TECHNOLOGY GROUP CO., LTD., BEIJING (CN)

(21) Appl. No.: 16/963,706

(22) PCT Filed: Mar. 3, 2020
(86) PCT No.: PCT/CN2020/077513

§ 371 Date: Jul. 21, 2020

(30) Foreign Application Priority Data
Mar. 11, 2019 (CN) 201910181877.8

(51) Int. Cl. A61B 8/02; A61B 8/04; A61B 8/08; G16H 50/30

(52) U.S. Cl. CPC A61B 8/02; G16H 50/30; A61B 8/488; A61B 8/04

(57) ABSTRACT

There is provided a method for determining a pulse transmission time, an arteriosclerosis detection apparatus and a system, the method comprising: receiving a single-lead electro-cardio signal; receiving a pulse wave signal of at least one of body parts, the pulse wave signal being detected by a micro ultrasonic detector arranged at the respective body parts; and determining the pulse transmission time by taking an R wave of the single-lead electro-cardio signal as a starting point and taking a feature point of the pulse wave signal of the at least one of the body parts as an end point.

### CROSS REFERENCE TO RELATED APPLICATIONS

This application is a U.S. National Stage Application under 35 U.S.C. § 371 of International Patent Application No. PCT/CN2020/077513, filed on Mar. 3, 2020, which claims priority from Chinese Patent Application No. 201910181877.8 filed on Mar. 11, 2019, the disclosures of which are incorporated herein by reference in their entirety.

### TECHNICAL FIELD

The present disclosure relates to a method for determining a pulse wave transmission time, an arteriosclerosis detection apparatus and a system.

### BACKGROUND

The morbidity and the mortality of cardiovascular and cerebrovascular diseases in China are still in an ascending stage, and the death of the cardiovascular diseases accounts for more than 40 percent of the death of resident diseases, which is at the first place and is far higher than that of tumors and other diseases. The arteriosclerosis detection apparatus is the important means for performing cardiovascular disease risk screening.

At present, the arteriosclerosis detection method determines a degree of arteriosclerosis mainly through blood pressure and electro-cardio signals and has an insufficient accuracy, whereas the arteriosclerosis detection apparatuses in the prior art mainly consist of modules such as a limb blood pressure measurement module, an electro-cardio detector and a heart sound module. Meanwhile, most of the arteriosclerosis detection apparatuses in the prior art provide optional Doppler ultrasound for measuring the transmission speed of the carotid artery, which results in that the entire apparatus has a large size, a high price and a relatively complex operation, and is not suitable for popularization in primary medical care and physical examination institutions.

### SUMMARY

According to a first aspect of the present disclosure, there is provided a method for determining a pulse transmission time, the method comprising: receiving a single-lead electro-cardio signal; receiving a pulse wave signal of at least one of body parts, the pulse wave signal being detected by a micro ultrasonic detector arranged at the respective body parts; and determining the pulse transmission time by taking an R wave of the single-lead electro-cardio signal as a starting point and taking a feature point of the pulse wave signal of the at least one of the body parts as an end point.

In some embodiments, the feature point of the pulse wave signal comprises at least one of a trough, a point of which a slope maximally rises with the trough as a base point, and a peak of a pulse wave.

In some embodiments, the at least one of the body parts comprises a limb.

According to a second aspect of the present disclosure, there is provided an arteriosclerosis detection apparatus, comprising: a communication interface configured to receive a single-lead electro-cardio signal and an ultrasonic signal of a pulse wave of at least one of body parts; a processor comprising a memory having computer-executable instructions stored thereon, wherein the computer-executable instructions, when executed by the processor, implement the method for determining the pulse transmission time according to any one of the embodiments in the first aspect of the present disclosure.

In some embodiments, the computer-executable instructions, when executed by the processor, further implement a step of: determining a pulse transmission speed from a heart to each of the body parts based on a pulse transmission time of each of the body parts and a distance between a sensing point of the single-lead electro-cardio signal and a sensing point of the pulse wave signal.

In some embodiments, the at least one of the body parts comprises a limb, and the computer-executable instructions, when executed the processor, further implement a step of: determining a blood pressure (BP) at respective parts of the limb according to the pulse transmission time (PTT) of the limb and a following formula:

$$BP = \frac{1}{γ} [ ln ( \frac{ρ {dS}^{2}}{{aE}_{0}} \} - 2 ln PTT ]$$

where γ is a quantity characterizing a feature of a blood vessel and has a numerical range of 0.016-0.018 mmHg−1; S is the distance between the sensing point of the single-lead electro-cardio signal and the sensing point of the pulse wave signal; E₀ is an elastic modulus when a pressure at the blood vessel wall is zero; BP is the blood pressure; PTT is the pulse transmission time; ρ indicates a density of blood; d indicates an inner diameter of the blood vessel; a is a coefficient related to individual characteristics and can be obtained by fitting actual measurement data.

An ankle-brachial index=SBPₐₙₖₗₑ/SBP_{upper arm} is determined according to the blood pressure at respective parts of the limb, wherein SBPₐₙₖₗₑ is a systolic pressure at an ankle, and SBP_{upper arm} is a systolic pressure at an upper arm.

In some embodiments, the computer-executable instructions, when executed by the processor, further implement a steps of: evaluating a degree of arteriosclerosis based on the blood pressure (BP) at the parts of the limb, the pulse transmission time (PTT) of the limb, a cardiac output amount (CO) per minute, and a peripheral resistance (TPR) as arteriosclerosis-related parameters, wherein

$$SV = 0.283 \text{/} {K}^{2} \times T \times ( {P}_{s} - {P}_{d} )$$

$${P}_{m} = \frac{1}{T} {\int }_{0}^{T} P ( t ) dt$$

$$TPR = {P}_{m} \text{/} CO$$

$$CO = SV \times 60 \text{/} T$$

$$K = ( {P}_{m} - {P}_{d} ) \text{/} ( {P}_{s} - {P}_{d} )$$

where SV is a cardiac output amount per stroke; K is a waveform value of a pulse wave; T is a period of the pulse wave; Pₛ is a systolic pressure; P_{d} is a diastolic pressure; Pₘ is a mean arterial pressure; CO is the cardiac output amount per minute; TPR is the peripheral resistance.

In some embodiments, the computer-executable instructions, when executing by the processor, further implement a step of: determining a damage index for each of the arteriosclerosis-related parameters to evaluate the degree of arteriosclerosis, the damage index (F) for each of the arteriosclerosis-related parameters being calculated using a following formula:

$$F = \{ \begin{matrix}1 - {2}^{\frac{V - RC}{α}} & , V > RC \\ 1 - {2}^{\frac{RF - V}{β}} & , V < RF \\ 0 & , RF \text{<<} V \text{<<} RC \end{matrix}$$

where V is an actual value of the arteriosclerosis-related parameter; RC is an upper limit of a normal range of the arteriosclerosis-related parameter; RF is a lower limit of the normal range of the arteriosclerosis-related parameter; F is the damage index for the arteriosclerosis-related parameter; α and β are constants and are obtained by fitting a data set of clinically-measured arteriosclerosis-related parameters and corresponding clinically-estimated damage indexes.

According to a third aspect of the present disclosure, there is provided an arteriosclerosis detection system, comprising the arteriosclerosis detection apparatus according to any one of the embodiments in the second aspect of the present disclosure, the system further comprising: a first slave comprising an electro-cardio detector configured to sense a single-lead electro-cardio signal of a user; a second slave comprising a micro ultrasonic detector configured to be worn on at least one of body parts of the user to detect an ultrasonic signal of a pulse wave thereof.

In some embodiments, the first slave further comprises: a first microprocessor configured to process the single-lead electro-cardio signal to obtain information of an R wave of the single-lead electro-cardio signal; a first communication circuit configured to transmit the information of the R wave of the single-lead electro-cardio signal; the second slave further comprises: a second microprocessor configured to process the ultrasonic signal of the pulse wave to obtain a feature point of the ultrasonic signal of the pulse wave; a second communication circuit configured to transmit the feature point of the ultrasonic signal of the pulse wave.

In some embodiments, each of the first slave and the second slave comprises a timer configured to determine first time information of the respective slaves; the arteriosclerosis detection apparatus transmits second time information to the first slave and the second slave via the communication interface; each of the first microprocessor and the second microprocessor is further configured to calculate a time deviation condition between the first time information and the second time information of the respective slaves; the first communication circuit and the second communication circuit are each further configured to transmit the respective time deviation information to the arteriosclerosis detection apparatus.

In some embodiments, the communication interface of the arteriosclerosis detection apparatus is further configured to receive the time deviation information; the processor of the arteriosclerosis detection apparatus is further configured to perform respective time compensations on signals transmitted by the first slave and the second slave according to the time deviation information.

In some embodiments, the micro ultrasonic detector is further configured to sense a blood vessel wall signal and a blood flow signal; the second microprocessor is further configured to acquire, based on the blood vessel wall signal and the blood flow signal, at least one of following parameters: an arterial elastic coefficient, a thickness of a blood vessel wall, and a viscosity of blood.

In some embodiments, the second slave comprises at least four second slaves configured to acquire the ultrasonic signal of the pulse wave of the limb, respectively.

In some embodiments, the arteriosclerosis detection system further comprises a power source configured to supply power to the arteriosclerosis detection system.

In some embodiments, the arteriosclerosis detection system further comprises a display configured to display evaluation information of the degree of arteriosclerosis.

It should be understood that both the foregoing general description and the following detailed description are only exemplary and explanatory, rather than limiting the present disclosure.

This section provides an overview of various implementations or examples of the technology described in the present disclosure, rather than a comprehensive disclosure of the full scope or all features of the disclosed technology.

### BRIEF DESCRIPTION OF THE DRAWINGS

To more clearly illustrate the technical solutions of the embodiments of the present disclosure, the drawings of the embodiments will be briefly introduced below, and it is obvious that the drawings in the following description only relate to some embodiments of the present disclosure, rather than limiting the present disclosure.

FIG. 1 is a flow chart of a method for determining a pulse transmission time according to embodiments of the present disclosure;

FIG. 2 is a schematic diagram of an R-wave interval of an electro-cardio signal as a reference and a pulse transmission time calculated by using a feature point of a pulse wave signal according to embodiments of the present disclosure;

FIG. 3 is a graph comparing the pulse wave transmission in two conditions of a good blood vessel and blood vessel sclerosis;

FIG. 4 is a schematic structural diagram of an arteriosclerosis detection apparatus according to embodiments of the disclosure;

FIG. 5 is a schematic diagram illustrating a relationship between the pulse transmission time and the pulse transmission speed and the degree of blood vessel sclerosis;

FIG. 6 is a waveform graph of a period of the pulse wave;

FIG. 7 is a schematic structural diagram of an arteriosclerosis detection system according to embodiments of the disclosure;

FIG. 8 is a schematic structural diagram of an arteriosclerosis detection system according to embodiments of the disclosure;

FIG. 9 is a flowchart of an operation of the arteriosclerosis detection system according to embodiments of the disclosure.

### DETAILED DESCRIPTION

In order to make objects, technical solutions and advantages of the embodiments of the present disclosure more apparent, the technical solutions of the embodiments of the present disclosure will be described clearly and completely below with reference to the drawings of the embodiments of the present disclosure. It is obvious that the described embodiments are only a few embodiments of the present disclosure, rather than all the embodiments. All other embodiments, which can be derived by a person skilled in the art from the described embodiments of the disclosure without inventive labors, are within the scope of protection of the present disclosure.

Unless defined otherwise, technical or scientific terms used herein shall have the ordinary meaning as understood by one of ordinary skill in the art to which the present disclosure belongs. The word “comprising” or “comprises” or the like means that an element or an item preceding the word comprises an element or an item listed after the word and equivalent thereof, but does not exclude other elements or items. The term “connected” or “coupled” or the like is not limited to physical or mechanical connections, but may comprise electrical connections, either directly or indirectly. The term “upper”, “lower”, “left”, “right” or the like is used only to indicate a relative positional relationship, and when the absolute position of the object being described is changed, the relative positional relationship may also be changed accordingly.

To make the following description of the embodiments of the present disclosure clear and concise, a detailed description of known functions and known components is omitted in the present disclosure.

With respect to the aforementioned technical problems exiting in the prior art, the present disclosure provides a method for determining a pulse wave transmission time, an arteriosclerosis detection apparatus and a system, which can determine the pulse transmission time accurately according to a single-lead electro-cardio signal and an ultrasonic signal of a pulse wave. The arteriosclerosis detection apparatus can be integrated with a single-lead electro-cardio detector and a micro ultrasonic pulse wave detection module to obtain the arteriosclerosis detection system, which can conveniently and accurately determine parameters comprising the pulse transmission time and the like which are important to the degree of arteriosclerosis, so as to further determine the degree of arteriosclerosis. The apparatus has a small volume, a low price, a convenient usage, and a high accuracy.

As compared with the prior art, the present disclosure has beneficial effects as follows.

The method for determining the pulse wave transmission time, and the arteriosclerosis detection apparatus and the system, as provided in the present disclosure, can determine the pulse transmission time accurately according to the single-lead electro-cardio signal and the ultrasonic signal of the pulse wave. The arteriosclerosis detection apparatus can be integrated with the single-lead electro-cardio detector and the micro ultrasonic pulse wave detection module to obtain the arteriosclerosis detection system, which can conveniently and accurately determine parameters comprising the pulse transmission time and the like which are important to the degree of arteriosclerosis, so as to further determine the degree of arteriosclerosis. The apparatus has a small volume, a low price, a convenient usage, and a high accuracy.

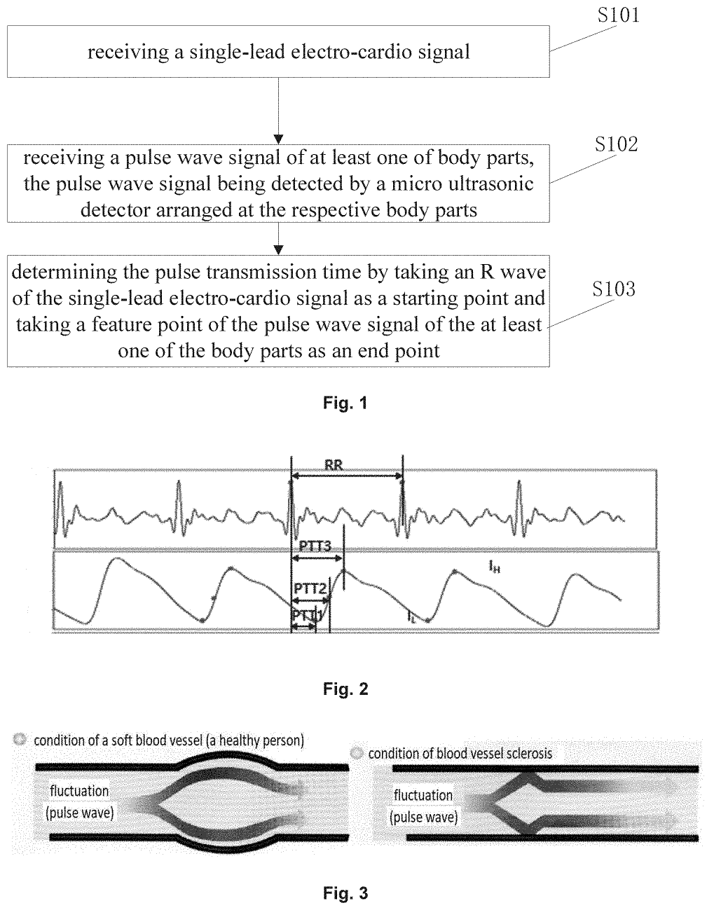

FIG. 1 is a flow chart of a method for determining a pulse transmission time according to embodiments of the present disclosure. As shown in FIG. 1, the present disclosure provides a method for determining a pulse transmission time, the method comprising steps S101 to S103.

At step S101, a single-lead electro-cardio signal is received. In some embodiments, the single-lead electro-cardio signal may be a lead electro-cardio signal of any one of a limb lead, a chest lead, and the like, which is not specifically limited herein. Different from multi-lead electro-cardio signals, some portable electro-cardio detection apparatuses (e.g., a smart wristwatch comprising an electro-cardio detection function) comprise a single-lead electro-cardio detector or have a function of collecting a single-lead electro-cardio signal. Thus, the single-lead electro-cardio signal can be received from a wide variety of portable electro-cardio detection apparatuses.

At step S102, a pulse wave signal of at least one of body parts is received, the pulse wave signal being detected by a micro ultrasonic detector arranged at the respective body parts, such as a wrist, a neck, an ankle of a user. The micro ultrasonic detector can adopt, for example, a miniaturized micro ultrasonic sensor, to accurately capture artery pulse conditions of a local measurement position via the Doppler ultrasound, and has a smaller size and is positioned more accurately in a time domain, as compared with other pulse wave signal detection apparatuses. Moreover, the micro ultrasonic sensor can be flexibly loaded at body end parts, such as limbs and the like, so that a sufficient distance between the sensing position of the pulse wave signal and the sensing position of the electro-cardio signal is ensured. This is beneficial to the reduction of disturbance of the signal processing errors on the pulse transmission time, so as to reduce the calculation error of the pulse transmission time.

At step S103, the pulse transmission time is determined by taking the R wave of the single-lead electro-cardio signal as a starting point and taking the feature point of the pulse wave signal of at least one of the body parts as an ending point. For example, after the single-lead electro-cardio signal and the pulse wave signal are received, the two signals are firstly preprocessed, such as noise filtering, baseline drift removal or the like, and then the information of the R wave of the single-lead electro-cardio signal and the feature point of the pulse wave signal are extracted. There are a variety of methods of extracting the information of the R wave of the electro-cardio signal and the feature point of the pulse wave signal, which are not specifically limited herein.

The method for determining the pulse wave transmission time as provided in the disclosure takes the R wave of the single-lead electro-cardio signal as the starting point and takes the feature point of the pulse wave signal of the body part detected by the micro ultrasonic detector as the end point, and starts timing from a starting point where the heart pumps blood, so that the pulse wave transmission time can be determined more accurately. Furthermore, the method utilizes the signals respectively collected by the single-lead electro-cardio detector and the micro ultrasonic detector which are more user-friendly to wear and use. Therefore, it is possible to accurately detect the pulse transmission time which is important to the degree of arteriosclerosis while the user experience is improved.

In some embodiments, the feature point of the pulse wave signal comprises at least one of a trough, a point of which a slope maximally rises with the trough as a base point, and a peak of the pulse wave. The R-wave interval (denoted as RR) of the electro-cardio signal as a reference, and the pulse transmission times (denoted as PTT1, PTT2 and PTT3, respectively) calculated by using, as the feature points, the trough, the point of which the slope maximally rises with the trough as the base point, and the peak of the pulse wave, respectively, are shown.

Illustratively, the pulse transmission time is calculated by using the peak of the pulse wave as the end point. The inventor has conducted a great number of clinical tests based on these three feature points, and as a result, found that, it is possible to derive a more accurate value of a blood voltage of the respective body parts from the pulse transmission time calculated by using the peak of the pulse wave as the end point (the derivation method will be described in detail hereinafter, and will not be repeated here), and it has the highest correlation with arteriosclerosis.

In some embodiments, the at least one of the body parts comprises a limb, so that a blood vessel condition of the limb of the user can be analyzed according to the pulse transmission time of the limb. Moreover, the limb is far away from the heart, which is beneficial to the reduction of disturbance of the signal processing errors on the pulse transmission time, so as to reduce the calculation error of the pulse transmission time.

For example, the pulse transmission time reflects the degree of blood vessel sclerosis to some extent. As shown in FIG. 3, blood vessels of a healthy person have good elasticity, and the pulse transmission time is relatively long. In case where the arterial blood vessel sclerosis occurs, the pulse transmission time is relatively short.

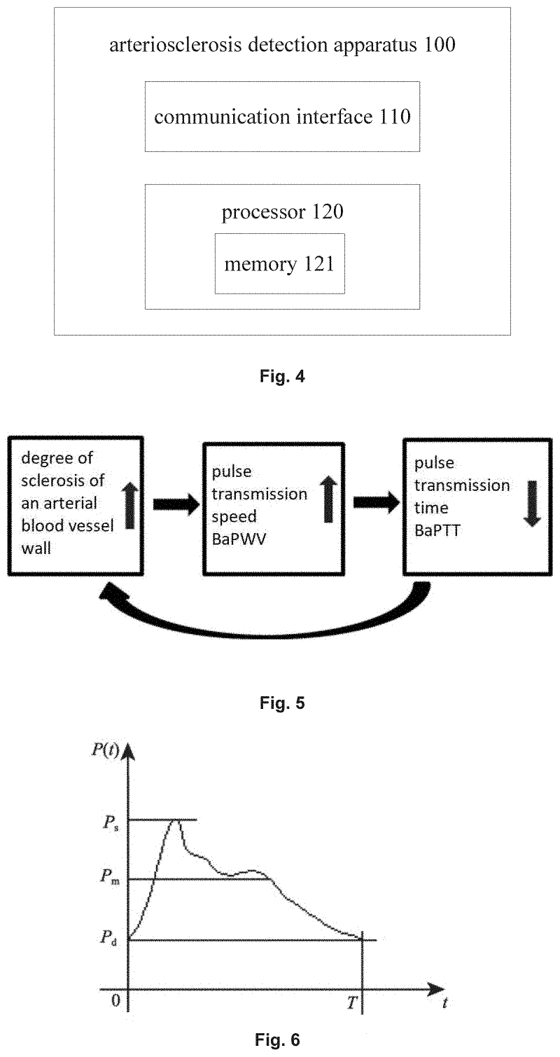

FIG. 4 is a schematic structural diagram of an arteriosclerosis detection apparatus according to embodiments of the disclosure. As shown in FIG. 4, the present disclosure provides an arteriosclerosis detection apparatus 100 which comprises a communication interface 110 and a processor 120, wherein the communication interface 110 is configured to receive a single-lead electro-cardio signal and an ultrasonic signal of a pulse wave of at least one of body parts, and the processor 120 further comprises a memory 121 on which computer-executable instructions are stored, the computer-executable instructions, when executed by the processor 120, implement the method for determining a pulse transmission time according to any one of the embodiments of the disclosure. For example, the single-lead electro-cardio signal and the ultrasonic signal of the pulse wave received by the communication interface 110 may be signals that have undergone the preprocessing or signals that have not undergone the preprocessing. If the two signals have not undergone the preprocessing, it is necessary for the processor 120 to firstly preprocess the single-lead electro-cardio signal and the ultrasonic signal of the pulse wave of the body part which are received by the communication interface 110, for example, noise removal or baseline drift removal, and then to extract the information of the R wave of the preprocessed single-lead electro-cardio signal and the feature point of the preprocessed pulse wave signal. Certainly, the preprocessing comprising the extraction of the information of the R wave and the feature point of the pulse wave signal may also be performed by using own processing units of the single-lead electro-cardio detector and the micro ultrasonic pulse detector, respectively.

For example, there are various methods for preprocessing and methods for extracting the information of the R wave of the single-lead electro-cardio signal and the feature point of the pulse wave signal, which are not specifically limited herein. Then, the computer-executable instructions stored on the memory 121 are executed to determine the pulse transmission time according to the information of the R wave of the single-lead electro-cardio signal and the feature point of the pulse wave signal, wherein the pulse transmission time has a high correlation with the degree of arteriosclerosis, so that the degree of arteriosclerosis of each of the body parts can be qualitatively determined according to the pulse transmission time.

The arteriosclerosis detection apparatus 100 as provided by the present disclosure can accurately determine the pulse transmission time based on the single-lead electro-cardio signal and the ultrasonic signal of the pulse wave, can conveniently and accurately obtain important parameters related to the degree of arteriosclerosis according to the determined pulse transmission time, and has a high accuracy in determining the degree of arteriosclerosis.

In some embodiments, the computer-executable instructions, when executed by the processor 120, further implement a step of: determining a pulse transmission speed from the heart to each of the body parts based on the pulse transmission time of each of the body parts and the distance between the sensing point of the single-lead electro-cardio signal and the sensing point of the pulse wave signal. For example, as can be derived from FIG. 5, the better the elasticity of the arterial vessel wall is, the slower the pulse transmission speed (baPWV) is; in the case that the sclerosis of the arterial vessel wall occurs, the pulse transmission speed (baPWV) will be increased accordingly; the faster the pulse transmission speed (baPWV) is, the higher the degree of the sclerosis of the arterial vessel wall is.

In some embodiments, the at least one of the body parts comprises a limb, so that the degree of arteriosclerosis of the limb is determined according to the pulse transmission time of the limb. The computer-executable instructions, when executed by the processor 120, further implement a step of: determining a blood pressure (BP) at respective parts of the limb according to the pulse transmission time (PTT) of the limb and a following formula:

$$BP = \frac{1}{γ} [ ln ( \frac{ρ {dS}^{2}}{{aE}_{0}} \} - 2 ln PTT ]$$

where γ is a quantity characterizing a feature of a blood vessel, and has a numerical range of 0.016-0.018 mmHg⁻¹, S is a distance between the sensing point of the single-lead electro-cardio signal and the sensing point of the pulse wave signal; E₀ is an elastic modulus when a pressure at the blood vessel wall is zero; BP is a blood pressure; PTT is the pulse transmission time; ρ indicates a density of blood; d indicates an inner diameter of the blood vessel; a is a coefficient related to individual characteristics and can be obtained by fitting actual measurement data. An ankle-brachial index=SBPₐₙₖₗₑ/SBP_{upper arm} is determined according to the blood pressure at respective parts of the limb, wherein ankle is SBPₐₙₖₗₑ is a systolic pressure at the ankle, and SBP_{upper arm} is a systolic pressure at the upper arm. For example, in the embodiments of the present disclosure, actual data of various parameters related to the blood vessel, such as the inner diameter (d) of the blood vessel, may be obtained through measurement by the micro ultrasonic pulse wave detector. The blood pressure at the parts of the limb obtained based on the actual data is more accurate than the blood pressure obtained by data modeling, so that the obtained ankle-brachial index is also more accurate. For example, the ankle-brachial index is used to evaluate the degree of arteriosclerosis of a lower limb, a value of the ankle-brachial index being greater than 1.30 characterizes arterial sclerosis, a value thereof being between 1.00 and 1.29 characterizes normal arteries, a value thereof being between 0.91 and 0.99 characterizes that the current artery is in the critical range of normality and sclerosis, a value thereof being between 0.41 and 0.90 characterizes that a user has a mild to moderate arterial disease, and a value thereof being between 0.00 and 0.40 characterizes that a user has a severe peripheral arterial disease.

In some embodiments, the computer-executable instructions, when executed by the processor 120, further implement a step of: evaluating a degree of arteriosclerosis based on the blood pressure at the parts of the limb, the pulse transmission time of the limb, a cardiac output amount (CO) per minute, and a peripheral resistance (TPR) as arteriosclerosis-related parameters, wherein

$$SV = 0.283 \text{/} {K}^{2} \times T \times ( {P}_{s} - {P}_{d} )$$

$${P}_{m} = \frac{1}{T} {\int }_{0}^{T} P ( t ) dt$$

$$TPR = {P}_{m} \text{/} CO$$

$$CO = SV \times 60 \text{/} T$$

$$K = ( {P}_{m} - {P}_{d} ) \text{/} ( {P}_{s} - {P}_{d} )$$

where SV is a cardiac output amount per stroke; K is a waveform value of the pulse wave; T is a period of the pulse wave; Pₛ is a systolic pressure; P_{d} is a diastolic pressure; Pₘ is a mean arterial pressure (as shown in FIG. 6); CO is the cardiac output amount per minute; TPR is the peripheral resistance.

For example, a magnitude of K depends on an area of a periodic waveform of the pulse wave and is a dimensionless value, and K will greatly change under different physiological states; the peripheral resistance (TPR) reflects a degree of patency of the blood vessel; the cardiac output amount (CO) per minute reflects an efficiency of the blood circulation system of the body. The degree of arteriosclerosis is comprehensively evaluated by combining the waveform value (K) of the pulse wave, the cardiac output amount (CO) per minute and the peripheral resistance (TPR), so that the evaluation of the degree of arteriosclerosis becomes more accurate. Sometimes, the degree of arteriosclerosis of a patient may be not significantly embodied on a certain arteriosclerosis-related parameter. By integrating these four arteriosclerosis-related parameters of the blood pressure (BP) of the limb, the pulse transmission time (PTT) of the limb, the cardiac output amount (CO) per minute and the peripheral resistance (TPR), the degree of arteriosclerosis can be comprehensively and accurately grasped to avoid missing inspection or error inspection.

In some embodiments, the computer-executable instructions, when executed by the processor 120, further implement a step of: determining a damage index (F) for each of the arteriosclerosis-related parameters to evaluate the degree of arteriosclerosis, the damage index for each of the arteriosclerosis-related parameters being calculated using a following formula:

$$F = \{ \begin{matrix}1 - {2}^{\frac{V - RC}{α}} & , V > RC \\ 1 - {2}^{\frac{RF - V}{β}} & , V < RF \\ 0 & , RF \text{<<} V \text{<<} RC \end{matrix}$$

where V is an actual value of the arteriosclerosis-related parameter; RC is an upper limit of a normal range of the arteriosclerosis-related parameter; RF is a lower limit of the normal range of the arteriosclerosis-related parameter, for example, when an average of the heart rate is 75 times per minute, the normal range of the cardiac output amount (CO) per minute is 4500 ml-6000 ml, that is, the upper limit (RC) of the normal range of the cardiac output amount (CO) per minute is 6000, and the lower limit (RF) thereof is 4500; F is the damage index for the arteriosclerosis-related parameter; α and β are constants and are obtained by fitting a data set of clinically-measured arteriosclerosis-related parameters and corresponding clinically-estimated damage indexes.

The inventor finds out through clinical comparative experiments that the damage index for each of the arteriosclerosis-related parameters calculated from the aforementioned definition of F has a high degree of correspondence with an actual degree of arteriosclerosis of the patient, and the larger the value of F is, the higher the risk of suffering from the arteriosclerosis disease is. By processing each of the arteriosclerosis-related parameters into the damage index, it is convenient for a user to quantitatively and intuitively determine the degree of arteriosclerosis. Furthermore, by continuously tracking the damage index for each of the arteriosclerosis-related parameters of the same user, the progress of the arteriosclerosis state can be accurately determined under a unified standard.

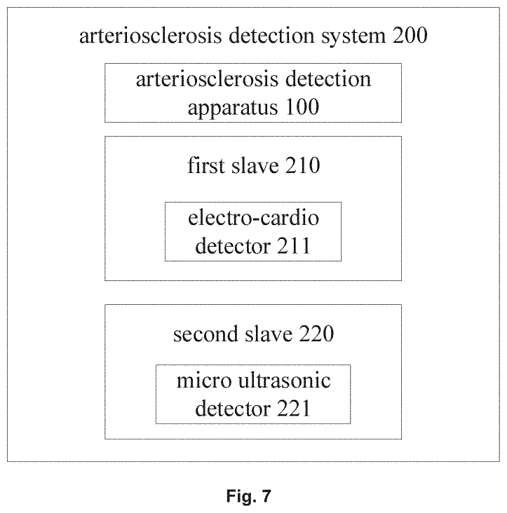

FIG. 7 is a schematic structural diagram of an arteriosclerosis detection system according to embodiments of the disclosure. As shown in FIG. 7, the present disclosure further provides an arteriosclerosis detection system 200 which comprises the arteriosclerosis detection apparatus 100 according to any one of the embodiments of the present disclosure, and the arteriosclerosis detection system 200 further comprises a first slave 210 and a second slave 220, wherein the first slave 210 comprises an electro-cardio detector 211 configured to sense a single-lead electro-cardio signal of a user, and the second slave 220 comprises a micro ultrasonic detector 221 configured to be worn on at least one of body parts of the user to detect an ultrasonic signal of a pulse wave of the body parts. For example, the first slave and the second slave further comprise a communication circuit (not shown in FIG. 7) that communicates with the arteriosclerosis detection apparatus 100 to transmit the sensed single-lead electro-cardio signal and the ultrasonic signal of the pulse wave to the arteriosclerosis detection apparatus 100.

For example, the image data acquired by the micro ultrasonic detector 221 is processed, so that not only the pulse wave signal can be detected, but also the various parameters related to the blood vessel, such as the inner diameter of the arterial blood vessel, can be obtained. The degree of arteriosclerosis can be evaluated based on the actual measurement data, and the obtained evaluation result has a higher accuracy. Moreover, when the pulse wave signal is detected by using the micro ultrasonic detector 221, it does not need to be bound on the user's body part to be detected, as a blood pressure measurement module in the existing arteriosclerosis apparatus, which can improve the comfort level of the user and is convenient and fast. The micro ultrasonic detector 221 has a small size and is integrated into the arteriosclerosis detection system 200, so that the arteriosclerosis detection system 200 has a small volume and a low price.

The arteriosclerosis detection system 200 provided by the present disclosure integrates the arteriosclerosis detection apparatus 100 with the single-lead electro-cardio detector and the micro ultrasonic pulse wave detector, which can conveniently and accurately determine parameters comprising a pulse transmission time and the like which are important to the degree of arteriosclerosis, so as to further determine the degree of arteriosclerosis. The system has a small volume, a low price, a convenient use, and a high accuracy.

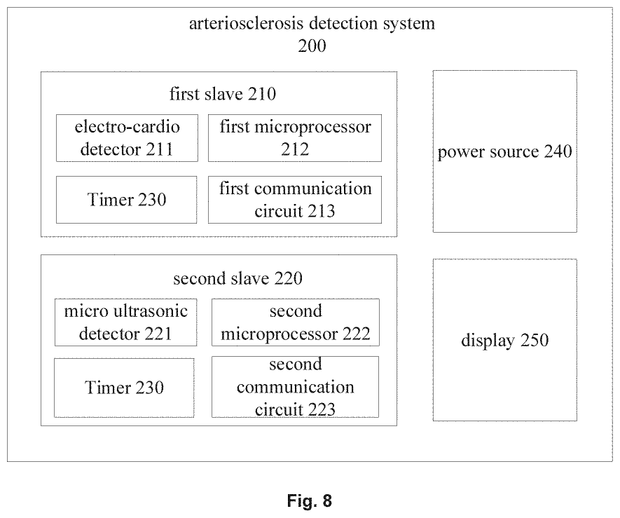

In some embodiments, as shown in FIG. 8, the first slave 210 further comprises a first microprocessor 212 and a first communication circuit 213, wherein the first microprocessor 212 is configured to process the sensed single-lead electro-cardio signal to obtain information of the R wave of the single-lead electro-cardio signal. For example, the first microprocessor 212 firstly preprocesses the single-lead electro-cardio signal, for example, removes myoelectric interference, power frequency interference, baseline drift, and then extracts the information of the R wave of the preprocessed single-lead electro-cardio signal. The first communication circuit 213 is configured to transmit the information of the R wave of the single-lead electro-cardio signal to the processor 120 of the arteriosclerosis detection apparatus 100. The second slave 220 further comprises a second microprocessor 222 and a second communication circuit 223, wherein the second microprocessor 222 is configured to process the detected ultrasonic signal of the pulse wave and extract the feature point of the preprocessed ultrasonic signal of the pulse wave. The second communication circuit 223 is configured to transmit the data of the feature point of the ultrasonic signal of the pulse wave to the processor 120 of the arteriosclerosis detection apparatus 100. Thus, the processor 120 determines the pulse transmission time according to the information of the R wave of the single-lead arteriosclerosis signal and the feature point of the ultrasonic signal of the pulse wave, so as to evaluate the degree of arteriosclerosis.

In some embodiments, as shown in FIG. 8, each of the first slave 210 and the second slave 220 includes a timer 230 configured to determine first time information of the respective slaves. The arteriosclerosis detection apparatus 100 transmits second time information to the first slave and the second slave via the communication interface 110. Each of the first microprocessor 212 and the second microprocessor 222 is further configured to calculate a time deviation condition between the first time information and the second time information of the respective slaves. Each of the first communication circuit 213 and the second communication circuit 223 is further configured to transmit the respective time deviation information to the arteriosclerosis detection apparatus 100. Specifically, the second time information is current time information of the arteriosclerosis detection apparatus 100, the first time information is current time information of the respective slaves, and the synchronization condition between the arteriosclerosis detection apparatus 100 and the respective slaves can be obtained according to the time deviation condition between the two time information.

In some embodiments, the communication interface 110 of the arteriosclerosis detection apparatus 100 is further configured to receive the time deviation information, and the processor 120 performs respective time compensations on signals transmitted from the first slave and the second slave according to the time deviation information, wherein the compensation manner can be to add a delay operation to the processor 120 to ensure the synchronization of operations between the arteriosclerosis detection apparatus 100 and the respective slaves.

In some embodiments, the micro ultrasonic detector 221 is further configured to sense a blood vessel wall signal and a blood flow signal; the second microprocessor 222 is further configured to acquire, based on the blood vessel wall signal and the blood flow signal, at least one of following parameters: an arterial elastic coefficient, a thickness of a vessel wall, and a viscosity of blood. For example, the arteriosclerosis condition can be better evaluated and the risk of suffering from the arteriosclerosis disease can be better predicted according to the parameters such as the arterial elastic coefficient, the thickness of the blood vessel wall, the viscosity of blood and the like in combination with the parameter of the degree of arteriosclerosis.

In some embodiments, the second slave 220 comprises at least four second slaves configured to acquire the ultrasonic signal of the pulse wave of the limb, respectively, so as to determine the degree of arteriosclerosis of the limb according to the pulse transmission time of the limb, and improve the accuracy of predicting the risk of suffering from the arteriosclerosis disease.

In some embodiments, as shown in FIG. 8, the arteriosclerosis detection system 200 further comprises a power source 240 configured to supply power to the arteriosclerosis detection system 200.

In some embodiments, as shown in FIG. 8, the arteriosclerosis detecting system 200 further comprises a display 250 configured to display evaluation information of the degree of arteriosclerosis. The display 250 can display the evaluation information of the degree of arteriosclerosis in various forms such as a graph and a table, so as to clearly and simply exhibit the evaluation information of the degree of arteriosclerosis to the user.

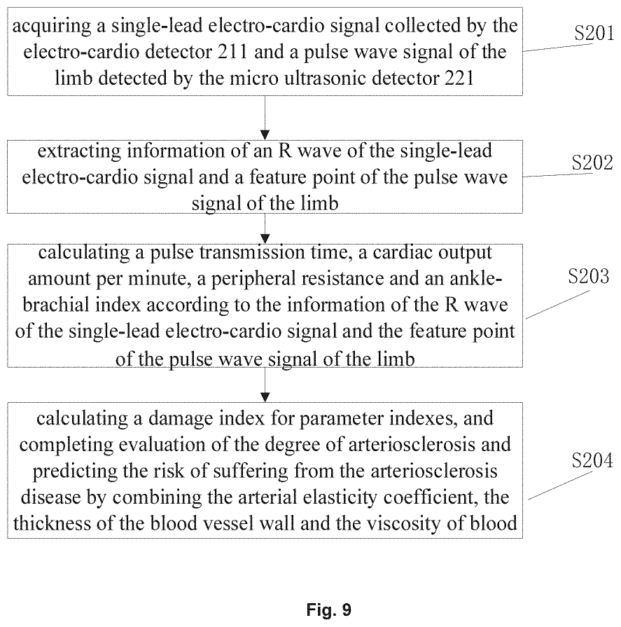

For example, an operation flow of the arteriosclerosis detection system 200 is shown in FIG. 9. At step S201, a single-lead electro-cardio signal collected by the electro-cardio detector 211 and a pulse wave signal of the limb detected by the micro ultrasonic detector 221 are acquired. At step S202, information of an R wave of the single-lead electro-cardio signal and a feature point of the pulse wave signal of the limb are extracted. At step S203, a pulse transmission time, a cardiac output amount per minute, a peripheral resistance and an ankle-brachial index are calculated according to the information of the R wave of the single-lead electro-cardio signal and the feature point of the pulse wave signal of the limb. At step S204, a damage index for the parameter indexes calculated at step S203 is calculated, and evaluation of the degree of arteriosclerosis is completed and the risk of suffering from the arteriosclerosis disease is predicted by combining the arterial elasticity coefficient, the thickness of the blood vessel wall and the viscosity of blood acquired by the micro ultrasonic detector 221.

The above description is intended to be illustrative rather than restrictive. For example, the above-described examples (or one or more solutions thereof) may be used in combination with each other. For example, other embodiments may be utilized by those of ordinary skill in the art upon reading the foregoing description. Moreover, in the foregoing detailed description, various features may be grouped together to simplify the disclosure. This should not be interpreted as an intention that a non-claimed disclosed feature is essential to any one of claims. Rather, the subject matter of the present disclosure may be less than all features of a particular disclosed embodiment. Thus, the following claims are hereby incorporated into the detailed description as examples or embodiments, wherein each of claims stands on its own as a separate embodiment, and it is contemplated that the embodiments can be combined with each other in various combinations or permutations. The scope of the disclosure should be determined with reference to the appended claims, along with the full scope of equivalents to which such claims are entitled.

The above embodiments are only exemplary embodiments of the present disclosure, and are not intended to limit the present disclosure, the scope of which is defined by the claims. A person skilled in the art may make various modifications or equivalent replacements to the disclosure within the spirit and protection scope of the disclosure, and such modifications or equivalent replacements should also be deemed to fall within the protection scope of the disclosure.

1. A method for determining a pulse transmission time, comprising:
receiving a single-lead electro-cardio signal;
receiving a pulse wave signal of at least one of body parts, the pulse wave signal being detected by a micro ultrasonic detector arranged at the respective body parts; and
determining the pulse transmission time by taking an R wave of the single-lead electro-cardio signal as a starting point and taking a feature point of the pulse wave signal of the at least one of the body parts as an end point.

2. The method for determining the pulse transmission time according to claim 1, wherein the feature point of the pulse wave signal comprises at least one of a trough, a point of which a slope maximally rises with the trough as a base point, and a peak of a pulse wave.

3. The method for determining the pulse transmission time according to claim 2, wherein the at least one of the body parts comprises a limb.

4. An arteriosclerosis detection apparatus, comprising:
a communication interface configured to receive a single-lead electro-cardio signal and an ultrasonic signal of a pulse wave of at least one of the body parts;
a processor comprising a memory having computer-executable instructions stored thereon, wherein the computer-executable instructions, when executed by the processor, implement the method for determining the pulse transmission time according to claim 1.

5. The arteriosclerosis detection apparatus according to claim 4, wherein the computer-executable instructions, when executed by the processor, further implement a step of:
determining a pulse transmission speed from a heart to each of the body parts based on the pulse transmission time of each of the body parts and a distance between a sensing point of the single-lead electro-cardio signal and a sensing point of the pulse wave signal.

6. The arteriosclerosis detection apparatus according to claim 5, wherein the at least one of the body parts comprises a limb, and the computer-executable instructions, when executed by the processor, further implement a step of:
determining a blood pressure (BP) at respective parts of the limb according to the pulse transmission time (PTT) of the limb and a following formula:
$$BP = \frac{1}{γ} [ ln ( \frac{ρ {dS}^{2}}{{aE}_{0}} \} - 2 ln PTT ]$$
where γ is a quantity characterizing a feature of a blood vessel and has a numerical range of 0.016-0.018 mmHg⁻¹; S is the distance between the sensing point of the single-lead electro-cardio signal and the sensing point of the pulse wave signal; E₀ is an elastic modulus when a pressure at a blood vessel wall is zero; BP is the blood pressure; PTT is the pulse transmission time; ρ indicates a density of blood; d indicates an inner diameter of the blood vessel; a is a coefficient related to individual characteristics and can be obtained by fitting actual measurement data;
determining an ankle-brachial index=SBP_{angle}/SBP_{upper arm} according to the blood pressure at the parts of the limb, wherein SBP_{angle} is a systolic pressure at an ankle, and SBP_{upper arm} is a systolic pressure at an upper arm.

7. The arteriosclerosis detection apparatus according to claim 6, wherein the computer-executable instructions, when executed by the processor, further implement a step of: evaluating a degree of arteriosclerosis based on the blood pressure (BP) at the parts of the limb, the pulse transmission time (PTT) of the limb, a cardiac output amount (CO) per minute, and a peripheral resistance (TPR) as arteriosclerosis-related parameters, wherein
$$SV = 0.283 \text{/} {K}^{2} \times T \times ( {P}_{s} - {P}_{d} )$$
$${P}_{m} = \frac{1}{T} {\int }_{0}^{T} P ( t ) dt$$
$$TPR = {P}_{m} \text{/} CO$$
$$CO = SV \times 60 \text{/} T$$
$$K = ( {P}_{m} - {P}_{d} ) \text{/} ( {P}_{s} - {P}_{d} )$$
where SV is a cardiac output amount per stroke; K is a waveform value of a pulse wave; T is a period of the pulse wave; Pₛ is a systolic pressure; P_{d} is a diastolic pressure; Pₘ is a mean arterial pressure; CO is the cardiac output amount per minute; TPR is the peripheral resistance.

8. The arteriosclerosis detection apparatus according to claim 7, wherein the computer-executable instructions, when executed by the processor, further implement a step of: determining a damage index for each of the arteriosclerosis-related parameters to evaluate the degree of arteriosclerosis, the damage index (F) for each of the arteriosclerosis-related parameters being calculated using a following formula:
$$F = \{ \begin{matrix}1 - {2}^{\frac{V - RC}{α}} & , V > RC \\ 1 - {2}^{\frac{RF - V}{β}} & , V < RF \\ 0 & , RF \text{<<} V \text{<<} RC \end{matrix}$$
where V is an actual value of the arteriosclerosis-related parameter; RC is an upper limit of a normal range of the arteriosclerosis-related parameter; RF is a lower limit of the normal range of the arteriosclerosis-related parameter; F is the damage index for the arteriosclerosis-related parameter; α and β are constants and are obtained by fitting a data set of clinically-measured arteriosclerosis-related parameters and corresponding clinically-estimated damage indexes.

9. An arteriosclerosis detection system comprising the arteriosclerosis detection apparatus according to claim 8, the arteriosclerosis detection system further comprising: a first slave comprising an electro-cardio detector configured to sense a single-lead electro-cardio signal of a user; a second slave comprising a micro ultrasonic detector configured to be worn on at least one of body parts of the user to detect an ultrasonic signal of a pulse wave thereof.

10. The arteriosclerosis detection system according to claim 9, wherein the first slave further comprises: a first microprocessor configured to process the single-lead electro-cardio signal to obtain information of an R wave of the single-lead electro-cardio signal; a first communication circuit configured to transmit the information of the R wave of the single-lead electro-cardio signal; the second slave further comprises: a second microprocessor configured to process the ultrasonic signal of the pulse wave to obtain a feature point of the ultrasonic signal of the pulse wave; a second communication circuit configured to transmit the feature point of the ultrasonic signal of the pulse wave.

11. The arteriosclerosis detection system according to claim 10, wherein each of the first slave and the second slave comprises a timer configured to determine first time information of the respective slaves; the arteriosclerosis detection apparatus transmits second time information to the first slave and the second slave via the communication interface; each of the first microprocessor and the second microprocessor is further configured to calculate time deviation information between the first time information and the second time information of the respective slaves; the first communication circuit and the second communication circuit are each further configured to transmit the respective time deviation information to the arteriosclerosis detection apparatus.

12. The arteriosclerosis detection system according to claim 11, wherein the communication interface of the arteriosclerosis detection apparatus is further configured to receive the time deviation information;
the processor of the arteriosclerosis detection apparatus is further configured to perform respective time compensations on signals transmitted by the first slave and the second slave according to the time deviation information.

13. The arteriosclerosis detection system according to claim 10, wherein the micro ultrasonic detector is further configured to sense a blood vessel wall signal and a blood flow signal; the second microprocessor is further configured to acquire, based on the blood vessel wall signal and the blood flow signal, at least one of following parameters: an arterial elastic coefficient, a thickness of a blood vessel wall, and a viscosity of blood.

14. The arteriosclerosis detection system according to claim 10, wherein the second slave comprises at least four second slaves configured to acquire the ultrasonic signal of the pulse wave of the limb, respectively.

15. The arteriosclerosis detection system according to claim 9, wherein the arteriosclerosis detection system further comprises a power source configured to supply power to the arteriosclerosis detection system.

16. The arteriosclerosis detection system according to claim 9, wherein the arteriosclerosis detection system further comprises a display configured to display evaluation information of the degree of arteriosclerosis.
